(19) United States
(12) Patent Application Publication
(10) Pub. No.: US 2014/0128613 A1
(43) Pub. Date: May 8, 2014

(54) A PROCESS FOR PREPARATION OF INTERMEDIATES OF DONEPEZIL HYDROCHLORIDE

(71) Applicants: Renugadevi Gurusamy, Mumbai (IN); Mita Roy, Mumbai (IN); Ramasubramanian Shanmuganathan, Mumbai (IN); Ashutosh Jagtap, Mumbai (IN); Sivaramakrishnan Hariharan, Mumbai (IN); Sushil Kumar Mishra, Mumbai (IN); Karuna Wankhede, Mumbai (IN)

(72) Inventors: Renugadevi Gurusamy, Mumbai (IN); Mita Roy, Mumbai (IN); Ramasubramanian Shanmuganathan, Mumbai (IN); Ashutosh Jagtap, Mumbai (IN); Sivaramakrishnan Hariharan, Mumbai (IN); Sushil Kumar Mishra, Mumbai (IN); Karuna Wankhede, Mumbai (IN)

(73) Assignee: PIRAMAL ENTERPRISES LIMITED, Mumbai (IN)

(21) Appl. No.: 14/006,160

(22) PCT Filed: Mar. 22, 2012
(86) PCT No.: PCT/IB12/51353

§ 371 Date: Sep. 19, 2013

(30) Foreign Application Priority Data
Mar. 25, 2011 (IN) 885/MUM/2011

(51) Int. Cl. C07D 213/50

(52) U.S. Cl. CPC C07D 213/50
USPC 546/340

(57) ABSTRACT

The present invention provides a process for the preparation of key intermediate for the synthesis 5,6-dimethoxy-2-[[1-(phenylmethyl)-4-piperidinyl]methyl]-1-indanone hydrochloride (donepezil hydrochloride). The present invention particularly provides a process for the preparation of 5,6-dimethoxy-2-(4-pyridylmethylene)-1-indanone comprising condensation of 5,6-dimethoxy-1-indanone with 4-pyridinecarboxaldehyde using an alkali metal hydroxide as a mild base in the presence of demineralized water as a solvent at a temperature in the range of 15° C. to 45° C. to yield 5,6-dimethoxy-2-(4-pyridylmethylene)-1-indanone, which is subsequently benzylated using benzyl bromide in the presence of solvent at a reflux temperature to yield 1-benzyl-4-[(5,6-dimethoxy-1-indanone-2-yl)methylene]pyridinium bromide.

### FIELD OF THE INVENTION

The present invention relates to a process for the preparation of key intermediates for the synthesis of 5,6-dimethoxy-2-[[1-(phenylmethyl)-4-piperidinyl]methyl]-1-indanone hydrochloride, hereinafter referred to as donepezil hydrochloride. More particularly, the present invention relates to a process for the preparation of 5,6-dimethoxy-2-(4-pyridylmethylene)-1-indanone and 1-benzyl-4-[(5,6-dimethoxy-1-indanone-2-yl)methylene]pyridinium bromide, the key intermediates for the synthesis of donepezil hydrochloride.

### BACKGROUND OF THE INVENTION

Donepezil hydrochloride is chemically known as 5,6-dimethoxy-2-[[1-(phenylmethyl)-4-piperidinyl]methyl]-1-indanone hydrochloride and it is structurally represented by the following formula I. It is an active ingredient of Aricept®, which is available in the market for oral administration as film-coated tablets.

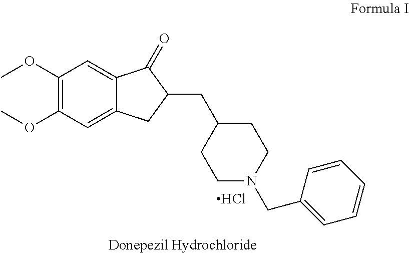 [SMILES: COc1cc2c(cc1OC)C(=O)C(CC1CCN(Cc3ccccc3)CC1)C2 ; Cl]

Donepezil is used to treat dementia associated with Alzheimer's disease, which is a brain disorder that affects the ability to remember, think clearly, communicate, and perform daily activities and may cause changes in mood and personality. Donepezil belongs to a class of medications called cholinesterase inhibitors, which improves mental function such as memory, attention, social interaction, reasoning and language abilities, and ability to perform activities of daily living, by increasing the amount of a certain naturally occurring substance in the brain. Donepezil may improve the ability to think and remember or slow the loss of these abilities in people who have Alzheimer's disease.

The prior art provides an enormous literature for the process of preparation of donepezil hydrochloride. The key intermediates for the preparation of donepezil hydrochloride are 5,6-dimethoxy-2-(4-pyridylmethylene)-1-indanone of formula IV (hereinafter referred to as, ‘the compound of formula IV’) and 1-benzyl-4-[(5,6-dimethoxy-1-indanone-2-yl)methylene]pyridinium bromide of formula V (herein after referred to as, ‘the compound of formula V’). Generally, the process for the preparation of the compound of formula V involves (i) condensation of 5,6-dimethoxy-1-indanone of formula II with 4-pyridinecarboxaldehyde of formula III using a strong base such as lithium diisopropylamide (LDA) or reagents such as p-toluenesulphonic acid in the presence of an organic solvent or mixtures thereof to yield 5,6-dimethoxy-2-(4-pyridylmethylene)-1-indanone of formula IV and (ii) benzylation of compound of formula IV using benzyl bromide in the presence of a solvent to yield compound of formula V.

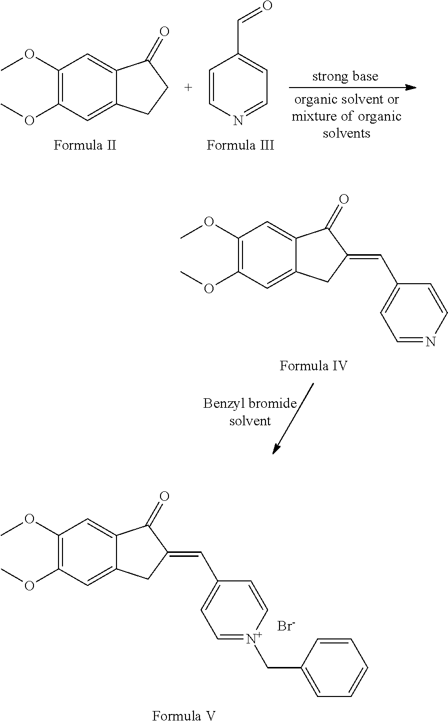

Donepezil and its pharmaceutically acceptable salts were first disclosed in the U.S. Pat. No. 4,895,841, wherein the process for the preparation of donepezil hydrochloride is disclosed. The process described in US '841 patent comprises the steps of: (i) reaction of 1-benzyl-4-piperidinecarboaldehyde with 5,6-dimethoxy-1-indanon-2-ylphosphonate in the presence of lithium diisopropylamide (LDA), (which is prepared in-situ by addition of a solution of n-butyllithium in hexane to the solution of diisopropylamine in anhydrous tetrahydrofuran (THF) at 0° C.) as a base at a temperature of −78° C., further the temperature of reaction mixture is raised to room temperature to yield 1-benzyl-4-(5,6-dimethoxy-1-indanon)-2-ylidenyl-methylpiperidine hydrochloride with 62% yield, (ii) reduction of the resulting compound as obtained in step (i) above using 10% palladium on carbon (Pd/C) in the presence of tetrahydrofuran (THF) as a solvent at room temperature under atmospheric pressure for 6 hours to obtain donepezil hydrochloride with 82% yield. The starting material, 5,6-dimethoxy-1-indanon-2-ylphosphonate of said reaction is prepared in-situ by reaction of 5,6-dimethoxy-1-indanone (compound of formula II) with hexamethyl-phosphoric amide in the presence of anhydrous THF.

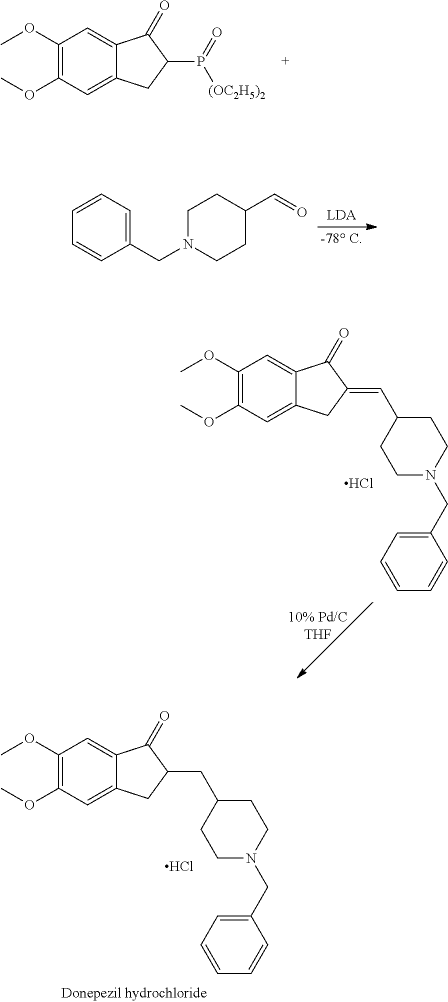

The process disclosed in said US'841 patent involves use of a strong base such as LDA, which is highly flammable liquid and reacts violently with water to give off flammable fumes. Moreover, LDA, on its combustion, emits hazardous by-products such as lithium oxide, carbon dioxide, carbon monoxide etc. Therefore, the use of such a hazardous compound as a base is neither safe nor eco-friendly and thus renders the process industrially not viable. Moreover, the in-situ preparation of LDA requires a very low temperature of −78° C., which is again industrially not viable.

U.S. Pat. No. 5,606,064 (and its equivalent EP Patent No. 711756) discloses a process for the preparation of donepezil. The process described in US '064 comprises the steps of: (i) reaction of 5,6-dimethoxy indanone of formula II with pyridine-4-carboxaldehyde of formula III using p-toluenesulphonic acid in the presence of toluene at reflux temperature for 5 hours to obtain 5,6-dimethoxy-2-(4-pyridylmethylene)-1-indanone of formula IV, yield 87%, (ii) benzylation of the compound of formula IV, obtained in step (i) above, using benzyl bromide in the presence of acetonitrile at reflux temperature yields 1-benzyl-4-[(5,6-dimethoxy-1-indanone-2-yl)methylene]pyridinium bromide of formula V, yield 83%, and (iii) reaction of the compound of formula V, obtained in step (ii) above, with platinum dioxide in the presence of methanol at a normal temperature and pressure for 24 hours yields donepezil, having yield of 81%. Although, the process of said US patent for the preparation of donepezil is practically applicable, it is industrially disadvantageous as in the step (i) of the above discussed process, the reaction requires a large volume of an organic solvent, for instance said process requires 13 volumes of toluene and furthermore, the reaction is carried out using p-toluenesulphonic acid, which is highly corrosive in nature. Moreover, the benzylation of compound of formula IV is carried out in the presence of 50 volumes acetonitrile as a solvent. The use of such large volumes of expensive solvents renders the process industrially not viable.

U.S. Pat. No. 7,148,354 describes a process for the preparation of donepezil. The process involves (i) refluxing 5,6-dimethoxy-1-indanone of formula II and pyridine-4-carboxaldehyde of formula III using p-toluenesulphonic acid in the presence of toluene as a solvent at a temperature ranging from 25° C. to 40° C. for 6 hours to yield 5,6-dimethoxy-2-(4-pyridylmethylene)-1-indanone of formula IV, yield 95.8%, (ii) reduction of the compound of formula IV, obtained in step (i) above, using 5% Pd/C in the presence of methanol and acetic acid as a solvent to yield 95.3% of 5,6-dimethoxy-2-piperidin-4-ylmethyl-1-indanone, and (iii) benzylation of product obtained in step (ii) above using benzyl bromide in the presence of ethanol as a solvent yields 92.3% of donepezil. The process of said US patent involves use of large quantity of the organic solvent in step (i) i.e. about 12.5 volumes of toluene, which is not cost-effective as well as not useful for the large scale manufacturing of the compound of formula IV. Moreover, use of a corrosive catalyst such as p—toluenesulphonic acid renders the process disadvantageous.

US Patent Application Publication No. 2007072905 discloses a process for the preparation of donepezil comprising the steps of: (i) reaction of 5,6-dimethoxy-1-indanone of formula II with pyridine-4-carboxaldehyde of formula III using p-toluenesulphonic acid in the presence of 104 volumes of toluene as the solvent at reflux temperature for 12 hours to yield 94% p-toluenesulphonic acid salt of the compound of formula IV, (ii) the product obtained in step (i) above is reduced using platinum dioxide in the presence of 75 volumes of methanol to obtain p-toluenesulphonic acid salt of 5,6-dimethoxy-2-((piperidin-4-yl)methyl)-1-indenone, and (iii) benzylation of the resulting compound as obtained in step (ii) above using benzyl bromide, N,N-dimethylformamide and potassium carbonate in the presence of water yields donepezil. The process disclosed in said US patent application suggests the use of very large quantity of the organic solvents, such as 104 volumes of toluene and 75 volumes of methanol, the use of such large volumes of solvent for commercial manufacturing renders the process costly and industrially not viable.

However, from the above discussion it is clearly apparent that, the processes disclosed in the prior art for the preparation of 5,6-dimethoxy-2-(4-pyridylmethylene)-1-indanone of formula IV necessitates use of a strong base such as lithium diisopropylamide (LDA) or reagents such as p-toluenesulphonic acid and large volumes of an organic solvent or mixture thereof. Thus, there is a need to develop a process for the preparation of key intermediates of donepezil hydrochloride, which is simple, cost-effective, industrially viable, non-hazardous and eco-friendly.

The inventors of the present invention have now found that the 5,6-dimethoxy-2-(4-pyridylmethylene)-1-indanone of formula IV can be obtained in good yield and high purity through an improved process involving condensation of 5,6-dimethoxy-1-indanone of formula II with 4-pyridinecarboxaldehyde of formula III using a mild base and demineralized water as a solvent, avoiding use of organic solvent and strong bases to yield 5,6-dimethoxy-2-(4-pyridylmethylene)-1-indanone. And further benzylation of the compound of formula IV using benzyl bromide in the presence of organic solvent yields 1-benzyl-4-[(5,6-dimethoxy-1-indanone-2-yl)methylene]pyridinium bromide of formula V. Subsequently 1-benzyl-4-[(5,6-dimethoxy-1-indanone-2-yl)methylene]pyridinium bromide is reduced using platinum dioxide, as disclosed in prior art references, to yield donepezil hydrochloride. Thus, the present invention provides a simple, cost-effective, industrially viable, non-hazardous and eco-friendly process for the preparation of the compound of formula IV and subsequently benzylation of compound of formula IV to obtain the compound of formula V, key intermediates for the synthesis of donepezil hydrochloride, which is used to treat dementia associated with Alzheimer's disease.

### OBJECTS OF THE INVENTION

An object of the present invention is to provide a process for the preparation of 5,6-dimethoxy-2-(4-pyridylmethylene)-1-indanone, the compound of formula IV.

Another object of the present invention is to provide an improved process for the benzylation of compound of formula IV to yield 1-benzyl-4-[(5,6-dimethoxy-1-indanone-2-yl)methylene]pyridinium bromide, the compound of formula V.

Another object of the present invention is to provide a process for the preparation of the compound of formula IV with yield≧97% and purity≧99%.

Yet another object of the present invention is to provide a process for the preparation of the compound of formula V with yield≧98% and purity≧97.5%.

Yet another object of the present invention is to provide a process for the preparation of the compound of formula IV using a mild base and demineralized water as a solvent, thereby avoiding use of large volumes of hazardous organic solvents and strong base such as lithium diisopropylamide (LDA) or reagents such as p-toluenesulphonic acid.

Yet another object of the present invention is to provide a process for the preparation of the compound of formula IV and subsequently benzylation of the compound of formula IV to obtain the compound of formula V, which is industrially viable, efficient and eco-friendly.

Still another object of the present invention is to provide a process for the preparation of the compound of formula IV and subsequently benzylation of compound of formula IV to obtain the compound of formula V, which is cost-effective.

### SUMMARY OF THE INVENTION

In accordance with the aspect of the present invention, there is provided a process for the preparation of 5,6-dimethoxy-2-(4-pyridylmethylene)-1-indanone of formula IV comprising condensation of 5,6-dimethoxy-1-indanone of formula II with 4-pyridinecarboxaldehyde of formula III using an alkali metal hydroxide as a mild base in the presence of demineralized water as a solvent at a temperature in the range of 15° C. to 45° C. to yield 5,6-dimethoxy-2-(4-pyridylmethylene)-1-indanone of formula IV.

In accordance with another aspect of the present invention, there is further provided an improved process for the benzylation of 5,6-dimethoxy-2-(4-pyridylmethylene)-1-indanone of formula IV using benzyl bromide in the presence of a solvent at a reflux temperature to yield 1-benzyl-4-[(5,6-dimethoxy-1-indanone-2-yl)methylene]pyridinium bromide of formula V.

The process of the present invention is schematically represented herein below:

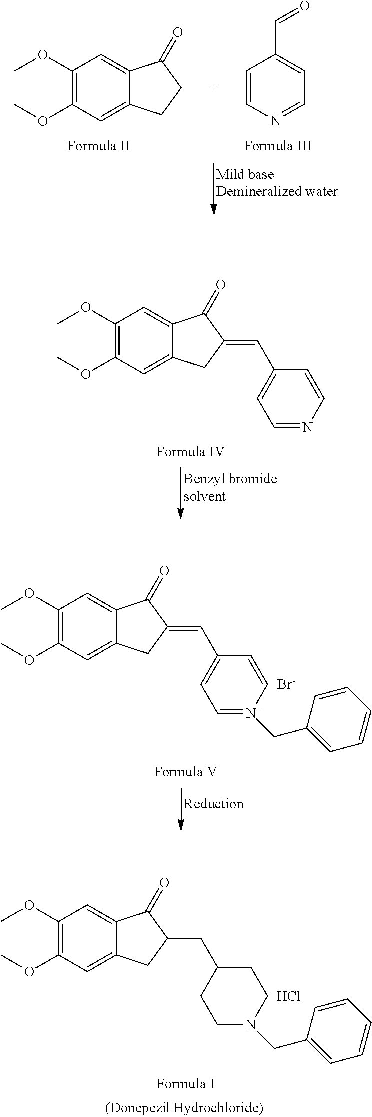

In accordance with another aspect of the present invention, there is provided a process for the preparation of the compound of formula IV having yield≧97% and purity≧99%, without any additional step of purification.

In accordance with another aspect of the present invention, there is provided a process for the preparation of the compound of formula V having yield≧98% and purity≧97.5%.

In accordance with yet another aspect of the present invention, the process of the present invention overcomes the disadvantages associated with the processes described in the cited prior art, which concerns with the use of strong bases such as lithium diisopropylamide or reagents such as p-toluenesulphonic acid. Also the process avoids use of an organic solvent as against the use of organic solvents in large volumes in the processes disclosed in the prior art, thereby making the instant process suitable for large scale manufacturing.

In accordance with further aspect of the present invention, the process for the preparation of 5,6-dimethoxy-2-(4-pyridylmethylene)-1-indanone of the compound of formula IV involves use of an alkali metal hydroxide such as potassium hydroxide or sodium hydroxide, which is a mild base and the reaction solvent, such as demineralized water. Thus, use of an alkali metal hydroxide as the base and demineralized water as the solvent renders the process of the present invention inexpensive and non-hazardous in comparison to the process of disclosed in the cited prior art references which involve use of a strong base such as lithium diisopropylamide (LDA) as well as the process involving use of organic solvents in large quantities. Accordingly, the process of the present invention is industrially viable, eco-friendly and cost-effective.

### DETAIL DESCRIPTION OF THE INVENTION

The present invention relates to a process for the preparation of 5,6-dimethoxy-2-(4-pyridylmethylene)-1-indanone of formula IV

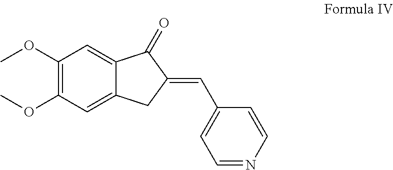 [SMILES: COc1cc2c(cc1OC)C(=O)/C(=C/c1ccncc1)C2]

comprising condensation of 5,6-dimethoxy-1-indanone of formula II,

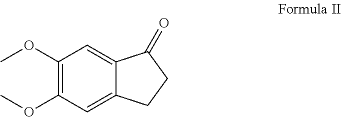

with 4-pyridinecarboxaldehyde of formula III,

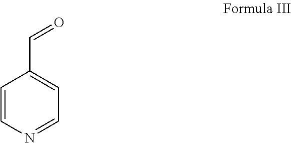

using an alkali metal hydroxide as a mild base in the presence of a solvent at a temperature in the range of 15° C. to 45° C. to obtain 5,6-dimethoxy-2-(4-pyridylmethylene)-1-indanone of formula IV, having yield≧97% and purity≧99%.

In accordance with the present invention, the condensation reaction is carried out using alkali metal hydroxide as a mild base is selected from potassium hydroxide or sodium hydroxide.

In accordance with the present invention, the condensation reaction of 5,6-dimethoxy-1-indanone of formula II with 4-pyridinecarboxaldehyde of formula III is carried out in the presence of demineralized water as a solvent, thereby avoids use of an organic solvents or mixtures thereof as a solvent.

In accordance with the present invention said condensation reaction is carried out using the compound of formula III in the mole equivalent of about 1.0 to 2.0 based on the compound of formula II.

In accordance with the present invention said condensation reaction is preferably carried out using the compound of formula III in the mole equivalent of 1.3 to 1.5 based on the compound of formula II.

In accordance with the present invention said condensation reaction is carried out using alkali metal hydroxide in the mole equivalent of about 0.75 to 2.0 based on the compound of formula II.

In accordance with the present invention said condensation reaction is preferably carried out using the alkali metal hydroxide in the mole equivalent of 0.9 to 1.2 based on the compound of formula II.

In accordance with the present invention, the condensation reaction of compound of formula II with compound of formula III is preferably carried out at a temperature in the range of 25° C. to 30° C.

The present invention further relates to an improved process for the preparation of 1-benzyl-4-[(5,6-dimethoxy-1-indanone-2-yl)methylene]pyridinium bromide of formula V.

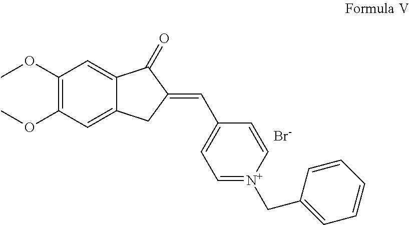 [SMILES: COc1cc2c(cc1OC)C(=O)/C(=C/c1cc[n+](Cc3ccccc3)cc1)C2 ; [Br-]]

The process for the preparation of compound of formula V comprises benzylation of 5,6-dimethoxy-2-(4-pyridylmethylene)-1-indanone of formula IV,

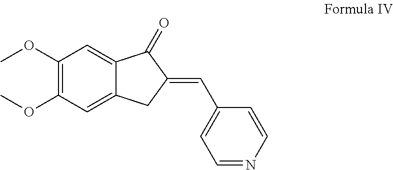 [SMILES: COc1cc2c(cc1OC)C(=O)/C(=C/c1ccncc1)C2]

using benzyl bromide in the presence of a solvent at reflux temperature to obtain 1-benzyl-4-[(5,6-dimethoxy-1-indanone-2-yl)methylene]pyridinium bromide, having yield≧98% and purity≧97.5%.

In accordance with the present invention the benzylation of compound of formula IV is carried out using 1 to 2 mole equivalent of benzyl bromide based on the compound of formula IV.

In accordance with the present invention the benzylation of compound of formula IV is carried out using methyl isobutyl ketone as a solvent.

In accordance with the present invention the benzylation of compound of formula IV is carried out using 12 volumes of methyl isobutyl ketone based on the compound of formula IV.

In accordance with the present invention the benzylation of compound of formula IV is carried out at a temperature ranging from 115° C. to 117° C.

The present invention further relates to a process for the preparation of 1-benzyl-4-[(5,6-dimethoxy-1-indanone-2-yl)methylene]pyridinium bromide of formula V comprising the steps of:
(a) condensation of 5,6-dimethoxy-1-indanone of formula II with 4-pyridinecarboxaldehyde of formula III using an alkali metal hydroxide as a mild base in the presence of a solvent at a temperature in the range of 15° C. to 45° C. to obtain 5,6-dimethoxy-2-(4-pyridylmethylene)-1-indanone of formula IV, having yield≧97% and purity≧99%, and
(b) benzylation of compound of formula IV, obtained in step (a) above using benzyl bromide in the presence of a solvent at reflux temperature to obtain 1-benzyl-4-[(5,6-dimethoxy-1-indanone-2-yl)methylene]pyridinium bromide of formula V, having yield≧98% and purity≧97.5%.

In an embodiment of the present invention the condensation reaction of 5,6-dimethoxy-1-indanone of formula II with 4-pyridinecarboxaldehyde of formula III is carried out using an alkali metal hydroxide as a mild base in the presence of demineralized water as the solvent to obtain 5,6-dimethoxy-2-(4-pyridylmethylene)-1-indanone of formula IV.

In accordance with the embodiment of the present invention the condensation reaction is carried out using alkali metal hydroxide as a mild base is selected from potassium hydroxide or sodium hydroxide.

In accordance with the embodiment of the present invention the condensation reaction of 5,6-dimethoxy-1-indanone of formula II with 4-pyridinecarboxaldehyde of formula III is carried out in the presence of demineralized water as a solvent, thereby avoids use of an organic solvents or mixtures thereof as a solvent.

In accordance with the embodiment of the present invention said condensation reaction is carried out using the compound of formula III in the mole equivalent of about 1.0 to 2.0 based on the compound of formula II.

In accordance with the embodiment of the present invention said condensation reaction is preferably carried out using the compound of formula III in the mole equivalent of 1.3 to 1.5 based on the compound of formula II.

In accordance with the embodiment of the present invention said condensation reaction is carried out using alkali metal hydroxide in the mole equivalent of about 0.75 to 2.0 based on the compound of formula II.

In accordance with the embodiment of the present invention said condensation reaction is preferably carried out using the alkali metal hydroxide in the mole equivalent of 0.9 to 1.2 based on the compound of formula II.

In accordance with the embodiment of the present invention, the condensation reaction of compound of formula II with compound of formula III is preferably carried out at a temperature in the range of 25° C. to 30° C.

In another embodiment of the present invention the compound of formula IV is benzylated using benzyl bromide in the presence of a solvent at reflux temperature to obtain 1-benzyl-4-[(5,6-dimethoxy-1-indanone-2-yl)methylene]pyridinium bromide of formula V.

In accordance with another embodiment of the present invention the benzylation of compound of formula IV is carried out using 1 to 2 mole equivalent of benzyl bromide based on the compound of formula IV.

In accordance with another embodiment of the present invention the benzylation of compound of formula IV is carried out using methyl isobutyl ketone as a solvent.

In accordance with another embodiment of the present invention the benzylation of compound of formula IV is carried out using 12 volumes of methyl isobutyl ketone based on the compound of formula IV.

In accordance with another embodiment of the present invention the benzylation of compound of formula IV is carried out at a temperature ranging from 115° C. to 117° C.

In accordance with the present invention, 5,6-dimethoxy-2-(4-pyridylmethylene)-1-indanone of formula IV prepared according to the process described herein below: To a round bottom flask charged demineralized water, 5,6-dimethoxy-1-indanone (the compound of formula II) and 4-pyridinecarboxaldehyde (the compound of formula III) and the resulting reaction mixture is stirred at a temperature of 15° C. to 45° C. for 5 minutes. To the reaction mixture a separately prepared solution of alkali metal hydroxide such as potassium hydroxide or sodium hydroxide in demineralized water is charged slowly over a period of 2 hours at a temperature ranging from of 15° C. to 45° C. The reaction mixture is further maintained at a temperature of 15° C. to 45° C. for 1 hour to obtain the product, 5,6-dimethoxy-2-(4-pyridylmethylene)-1-indanone (the compound of formula IV). At this stage the reaction mixture is analyzed using high performance liquid chromatography (HPLC) to monitor the completion of reaction. The product obtained is then filtered and washed with demineralized water. Dry the product under vacuum at a temperature of 50° C. to 55° C. for 10 hours. The yield of the product is ≧98% and the purity of the product is ≧99%. The compound of formula IV as obtained above is sufficiently pure to use it as such in the next step, without any additional step of purification.

The compound of formula IV, 5,6-dimethoxy-2-(4-pyridylmethylene)-1-indanone prepared using the above process may benzylated to obtain 1-benzyl-4-[(5,6-dimethoxy-1-indanone-2-yl)methylene]pyridinium bromide of formula V. The compound of formula V prepared according to the process described herein below. To a round bottom flask charged methyl isobutyl ketone and 5,6-dimethoxy-2-(4-pyridylmethylene)-1-indanone and reflux the reaction mixture at a temperature of 115° C. to 117° C. To the refluxing reaction mixture then dropwise added benzyl bromide over a period of 15 minutes. The reaction mixture further stirred for 2.5 hours. At this stage the reaction mixture is analyzed using HPLC to monitor completion of reaction. Cool the reaction mixture at a temperature of 25° C.-28° C. to obtain a solid. Filter the solid obtained and washed with methyl isobutyl ketone. Dry the solid under vacuum at a temperature of 50° C. to 55° C. for 1-2 hours to yield 1-benzyl-4-[(5,6-dimethoxy-1-indanone-2-yl)methylene]pyridinium bromide.

It is thus a possible by the way of the present invention to achieve the much desired synthesis for the preparation of key intermediates of donepezil hydrochloride of formula I.

The staring material of the process of the present invention such as 5,6-dimethoxy-1-indanone of formula II and 4-pyridinecarboxaldehyde of formula III are made available from the commercial sources.

As previously discussed the compound of formula V, 1-benzyl-4-[(5,6-dimethoxy-1-indanone-2-yl)methylene]pyridinium bromide may be further converted to the donepezil or its pharmaceutically acceptable salts, which is used to treat dementia associated with Alzheimer's disease. The compound of formula V obtained using the process of the present invention may be converted to donepezil by following the process described in the cited prior art, U.S. Pat. No. 5,606,064, which is incorporated herein by reference. Mainly, the compound of formula V as obtained by the process of the present invention is hydrogenated using platinum dioxide in the presence of methanol at normal pressure and at room temperature to yield 5,6-dimethoxy-2-[[1-(phenylmethyl)-4-piperidinyl]methyl]-1-indanone, that is donepezil.

The following examples which fully illustrate the practice of the preferred embodiments of the present invention are intended to be for illustrative purpose only and should not be constructed in anyway to limit the scope of the present invention.

### EXAMPLES

### Example 1

### Process for preparation of 5,6-dimethoxy-2-(4-pyridylmethylene)-1-indanone of formula IV

To a 1 liter round bottom flask equipped with a mechanical stirrer, thermometer pocket, addition funnel and a condenser, charged demineralized water (250 ml), 5,6-dimethoxy-1-indanone (25 g) and pyridine-4-carboxaldehyde (19.5 g). The reaction mixture was stirred for 5 minutes at a temperature of 25° C. to 30° C. To the reaction mixture then charged a solution of potassium hydroxide (5.2 g) dissolved in demineralized water (125 ml) slowly over period of 2 hours at a temperature of 25° C. to 30° C. The reaction mixture was further maintained at a temperature of 25° C. to 30° C. for 1 hour to obtain the product, 5,6-dimethoxy-2-(4-pyridylmethylene)-1-indanone. The product obtained was then filtered and washed with demineralized water. Dry the product under vacuum at a temperature of 50° C. to 55° C. for 10 hours.

Yield: 98%

Purity: 99.71%

### Example 2

### Process for preparation of 5,6-dimethoxy-2-(4-pyridylmethylene)-1-indanone of formula IV

To a 3 liter round bottom flask equipped with a mechanical stirrer, thermometer pocket, addition funnel and a condenser, charged demineralized water (1000 ml), 5,6-dimethoxy-1-indanone (100 g) and pyridine-4-carboxaldehyde (78.1 g). The reaction mixture was stirred for 5 minutes at a temperature of 25° C. to 30° C. To the reaction mixture then charged a solution of sodium hydroxide (20.8 g) dissolved in demineralized water (500 ml) slowly over period of 4 hours at a temperature of 25° C. to 30° C. The reaction mixture was further maintained at a temperature of 25° C. to 30° C. for 1 hour to obtain the product, 5,6-dimethoxy-2-(4-pyridylmethylene)-1-indanone. The product obtained was then filtered and washed with demineralized water. Dry the product under vacuum at a temperature of 50° C. to 55° C. for 10 hours

Yield: 97.5%

Purity: 99.5%

### Example 3

### Process for preparation of 1-benzyl-4-[(5,6-dimethoxy-1-indanone-2-yl)methylene]pyridinium bromide of formula V

To a round bottom flask equipped with mechanical stirrer, thermometer pocket, addition funnel and condenser, charged methyl isobutyl ketone (900 ml) and 5,6-dimethoxy-2-(4-pyridylmethylene)-1-indanone (75 g). The reaction mixture was then refluxed at a temperature of 115° C. to 117° C. To the refluxing reaction mixture then added benzyl bromide (56.6 g) dropwise over a period of 15 minutes. The reaction mixture stirred further for 2.5 hours. Cooled the reaction mixture at a temperature of 25° C. to 28° C. to obtain the product, 1-benzyl-4-[(5,6-dimethoxy-1-indanone-2-yl)methylene]pyridinium bromide. Filter the product obtained and washed with methyl isobutyl ketone. Dry the product under vacuum at a temperature of 50° C. to 55° C. for 1-2 hours.

Yield: 98%

Purity: 98.21%

### Example 4

### 1-benzyl-4-[(5,6-dimethoxy-1-indanone-2-yl)methylene]pyridinium bromide of formula V

To a round bottom flask equipped with mechanical stirrer, thermometer pocket, addition funnel and condenser, charged methyl isobutyl ketone (1200 ml) and 5,6-dimethoxy-2-(4-pyridylmethylene)-1-indanone (100 g). The reaction mixture was then refluxed at a temperature of 115° C. to 117° C. To the refluxing reaction mixture then added benzyl bromide (75.5 g) dropwise over a period of 30 minutes. The reaction mixture stirred further for 2.5 hours. Cooled the reaction mixture at a temperature of 25° C. to 28° C. to obtain the product, 1-benzyl-4-[(5,6-dimethoxy-1-indanone-2-yl)methylene]pyridinium bromide. Filter the product obtained and washed with methyl isobutyl ketone. Dry the product under vacuum at a temperature of 50° C. to 55° C. for 1-2 hours.

Yield: 98.76%

Purity: 97.5%

Analytical Method of Analysis
HPLC column: Zorbax Extended C-18, 4.6×250 mm, 5 μm
Detector: UV 230 nm
Mobile phase (MP): MP A-Buffer, MP B-ACN
Buffer preparation: 1 ml of phosphoric acid in 1 L of water, pH adjusted to 6.5 using triethanylamine
Injection volume: 10 μL
Oven temperature: 50° C.
Flow rate: 1.0 ml/minutes
Diluent: water:ACN (1:1)
Gradient:

| Time (min) | Mobile Phase A (%) | Mobile Phase B (%) |
|---|---|---|
| 0 | 90 | 10 |
| 15 | 70 | 30 |
| 15.2 | 60 | 40 |
| 30 | 50 | 50 |
| 35 | 90 | 10 |
| 40 | 90 | 10 |

1. A process for the preparation of 5,6-dimethoxy-2-(4-pyridylmethylene)-1-indanone of formula I 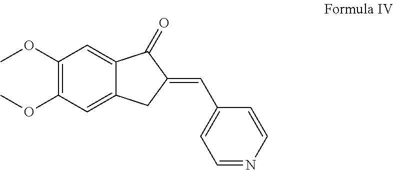 [SMILES: COc1cc2c(cc1OC)C(=O)/C(=C/c1ccncc1)C2]
comprising condensation of 5-dimethoxy-1-indanone of formula II 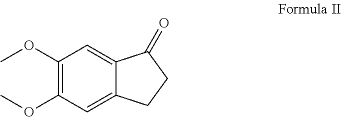
with 4-pyridinecarboxaldehyde of formula III 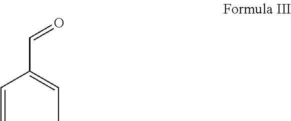
using an alkali metal hydroxide as a mild base in the presence of a solvent at a temperature in the range of 15° C. to 45° C. to obtain 5,6-dimethoxy-2-(4-pyridylmethylene)-1-indanone of formula IV, having purity>99%.

2. A process as claimed in claim 1 further comprising the step of benzylation of 5,6-dimethoxy-2-(4-pyridylmethylene)-1-indanone of formula IV using benzyl bromide in the presence of a solvent at a reflux temperature to obtain 1-benzyl-4-[(5,6-dimethoxy-1-indanone-2-yl)methylene]pyridinium bromide of formula V, having purity>97.5%.

3. The process as claimed in claim 1, wherein said alkali metal hydroxide is selected from potassium hydroxide or sodium hydroxide.

4. The process as claimed in claim 1, wherein the alkali metal hydroxide is used in the mole equivalent of about 0.75 to 2.0 based on the compound of formula II.

5. The process as claimed in claim 4, wherein said alkali metal hydroxide is used in the mole equivalent of 0.9 to 1.2 based on the compound of formula II.

6. The process as claimed in claim 1, wherein said condensation of compound of formula II with the compound of formula III is carried out in the presence of demineralized water as a solvent.

7. The process as claimed in claim 1, wherein the compound of formula III is used in the mole equivalent of about 1.0 to 2.0 based on the compound of formula II.

8. The process as claimed in claim 7, wherein said compound of formula III is used in the mole equivalent of 1.3 to 1.5 based on the compound of formula II.

9. The process as claimed in claim 1, wherein said condensation is carried out at a temperature in the range of 25° C. to 30° C.

10. The process as claimed in claim 2, wherein said benzylation is carried out in the presence of methyl isobutyl kctonc as a solvent.

11. The process as claimed in claim 2, wherein said benzylation is carried out using 1 to 2 mole equivalent of benzyl bromide based on the compound of formula IV.

12. The process as claimed in claim 2, wherein said benzylation is carried out using 12 volumes of methyl isobutyl ketone based on the compound of formula IV.

13. The process as claimed in claim 2, wherein said benzylation is carried out at a temperature ranging from 115° C. to 117° C.

14-15. (canceled)
